(19) United States
(12) Plant Patent Application Publication
(10) Pub. No.: US 2002/0092073 P1
(43) Pub. Date: Jul. 11, 2002

(54) Chrysanthemum named AURORA

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 09/734,656

(22) Filed: Dec. 13, 2000

(51) Int. Cl. A01H 5/00

(52) U.S. Cl. PLT/294

(57) ABSTRACT

A chrysanthemum plant named AURORA characterized by its medium sized blooms with white ray florets and prolific branching; natural season flower date September 11-16; blooming for a period of 5 weeks.

### BACKGROUND OF THE INVENTION

The new plant of the present invention comprises a new and distinct cultivar of Chrysanthemum plant that is a cross of unknown Dendranthema Grandiflora parents. The new and distinct cultivar was a seedling resulting from the open pollination among groups of chrysanthemum cultivars maintained under the control of the inventor for breeding purposes. The new and distinct cultivar was discovered and selected as one flowering plant by Rob Noodelijk on a cultivated field in Rijsenhout Holland in September 1998. The plant has been asexually reproduced by cuttings in greenhouses at Rijsenhout Holland. The new cultivar has been found to retain its distinctive characteristics through successive propagations.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

FIG. 2 shows the various stages of bloom and foliage of the new cultivar.

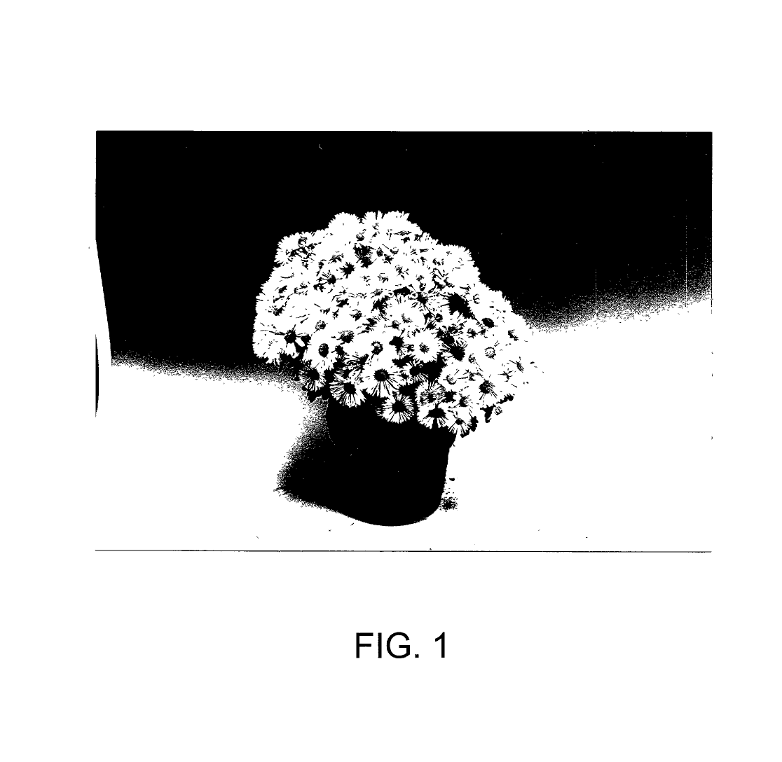

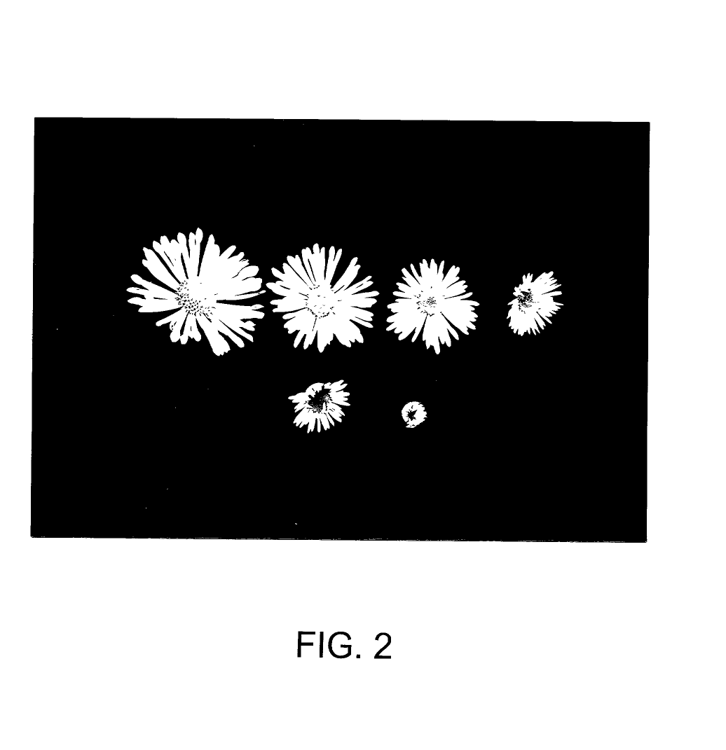

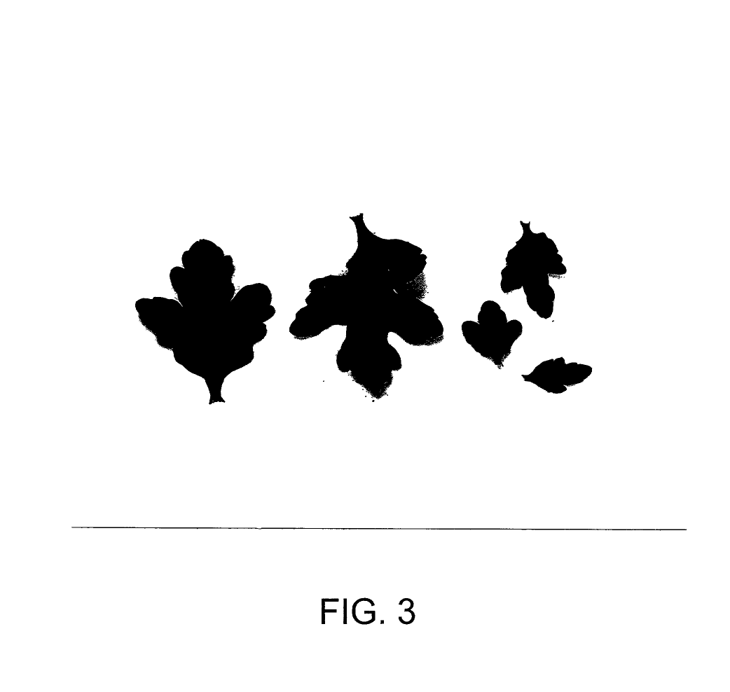

### DESCRIPTION OF THE INVENTION

This new variety of chrysanthemum is of the botanical classification Dendranthema Grandiflora. The observations and measurements were gathered from plants grown out door in Rijsenhout, Holland under natural day length and temperature and planted week 22 in 1999 and 2000. The natural blooming date of this crop was September 11-16 (week 37). The average height of the plants was 36-40 cms. This new variety produces medium sized blooms with white ray florets and yellow disc-florets blooming for a period of 5 weeks.

The following is a description of the plant and characteristics that distinguish AURORA as a new and distinct variety.

The color designations are taken from the plant itself. Accordingly, any discrepancies between the color designations and the colors depicted in the photographs are due to photographic tolerances.

Botanical classification: Cultivar aurora

Bud.—

Size.—small, cross-section+/−0.9 cm height +/−0.7 cm.

Outside color.—Yellow-white 158C.

Bloom.—

Size.—Medium.

Fully expanded.—5.5-6.0 cm.

Borne (number of blooms per branch).—Upper and lower portion plural blooms per branch (approx. 4 -5 blooms per branch).

Length of lateral branch.—From top till bottom 16-17 cm.

Lateral branch, attachment.—Strong.

branching.—Prolific with 6-8 breaks after pinching.

Form.—Daisy (with tubular petals).

Performance (on the plant) 5 weeks.

Color.—

Center of flower.—Immature Yellow-green 153D

Mature yellow 12A.

Color of the upper surface of the majority of the ray-florets.—White 155D.

Color of the lower surface of the majority of the ray-florets.—White 155D.

Tonality from distance.—A daisy garden mum with white blooms.

Discoloration.—None.

Ray florets.—

Texture.—Upper side and under side smooth.

Number.—65-68.

Cross-section.—Tubular.

Longitudinal axis of the majority.—Straight.

Shape of end.—Spatulate.

Fragrance.—Typical chrysanthemum.

Disc diameter (natural season crop when visible).—1.5 cm.

Reproductive organs.—

Stamen (present in disc florets only).—Yellow, thick, +/−4 mm length.

Pollen.—Present.

Styles (present in both ray and disc florets).—Green, thin.

Style length.—+/−4 mm.

Stigmas.—Yellow-green.

Stigma width.—+/−1 mm.

Ovaries.—Enclosed in calyx.

Plant.—

Form.—A garden mum (pot mum) meant for out door use.

Growth.—Vigorous.

Height (from soil level).—36-40 cm.

Stem color.—Green 138C.

Stem strength.—Strong.

Stem brittleness.—Present.

Stem anthocyanin coloration.—Absent.

Natural blooming date.—September 11-16.

Foliage.—

Color.—Upper side green 146A Under side green 147C.

Size.—Very small, length +/−4.5 cm width +/−3.5 cm.

Quantity (number per lateral branch).—8-10.

Shape.—Round.

Texture.—Fleshy.

Ribs and veins.—Ribs and veins not so well developed.

Edge.—Lobed.

Shape of base of sinus between lateral lobes.—Acute.

Margin of sinus between lateral lobes.—Diverging.

Shape of base.—Asymmetrical.

Apex.—Cuspidate.

I claim:

1. A new and distinc variety of chrysanthemum plant as described and illustrated
